(19) United States
(12) Patent Application Publication
(10) Pub. No.: US 2024/0091326 A1
(43) Pub. Date: Mar. 21, 2024

(54) Treatment of Glaucoma by Neuroprotection

(71) Applicant: The Regents of the University of California, Oakland, CA (US)

(72) Inventors: Lu Chen, Berkeley, CA (US); Lingling Zhang, Berkeley, CA (US)

(73) Assignee: The Regents of the University of California, Oakland, CA (US)

(21) Appl. No.: 18/518,981

(22) Filed: Nov. 25, 2023

Related U.S. Application Data
(63) Continuation of application No. PCT/US22/33699, filed on Jun. 16, 2022.
(60) Provisional application No. 63/212,595, filed on Jun. 18, 2021.

(51) Int. Cl. A61K 38/55; A61K 9/00; A61P 27/06

(52) U.S. Cl. CPC A61K 38/55; A61K 9/0048; A61P 27/06

(57) ABSTRACT

Ocular serpinA3 activity is modulated to treat glaucoma by neuroprotection, comprising administering to an eye in need thereof a serpinA3 polypeptide or a nucleic acid encoding the serpinA3 polypeptide.

This invention was made with government support under EY028995 awarded by the National Institutes of Health. The government has certain rights in the invention.

### INTRODUCTION

Glaucoma is a disease of optic neuropathy that affects at least 60 million people worldwide. It is a disease of optic neuropathy characterized by optic nerve damage and retinal ganglion cell (RGC) death. Currently, there is no cure for glaucoma, and there is a lack of neuroprotective drug for glaucoma therapy. Existing approaches, such as eye drops, laser and surgeries to lower intraocular pressure, are of limited efficacy and have many side effects as well. SerpinA3/Alpha-1-Antichymotrypsin is a known serine-proteinase inhibitor, class A, member 3.

### SUMMARY OF THE INVENTION

This invention provides methods and compositions to modulate ocular Serpina3n activity to treat glaucoma by neuroprotection.

In an aspect the invention provides a method of treating glaucoma, comprising locally or systemically administering to an eye in need thereof a serpinA3 polypeptide or a nucleic acid encoding the serpinA3 polypeptide.

In embodiments:
the administering step comprises delivering the serpinA3 polypeptide;
the administering step comprises delivering the nucleic acid encoding serpinA3 polypeptide;
the administering is systemically administering, such as intravenously;
the administering is locally administering, such as by eyedrop, intracameral, subconjunctival, subretinal, intrascleral, intravitreal, or retrobulbar injection;
the administering step comprises delivery by eye drop;
the administration step comprises topical delivery in an ophthalmic gel, ointment, suspension or solution;
further comprising treating the eye (or a person comprising the eye) a second, different glaucoma treatment;
further comprising treating the eye (or a person comprising the eye) a second, different glaucoma treatment, selected from eye drops, systemic (e.g. oral) medication, laser, and surgery to prevent or treat glaucoma;
further comprising detecting a resultant amelioration of the glaucoma; and/or
further comprising detecting a resultant amelioration of the glaucoma by a decrease in RGC death or axonal damage.

In an aspect the invention provides an ophthalmic formulation of a serpinA3 polypeptide or a nucleic acid encoding the serpinA3polypeptide, configured for treating glaucoma, wherein optionally:

in the form of a topical ophthalmic gel, ointment, suspension or solution;
the dosage form is a serpinA3-loaded contact lens, eye drop, depot or bollus;
packaged in an eye drop dispenser;
loaded in a syringe configured for intracameral, subconjunctival, subretinal, intrascleral, intravitreal, or retrobulbar injection; and/or
further comprising excipients and features suitable for direct, topical delivery to the eye, selected from the group consisting of opthalmically suitable clarity, pH buffer, tonicity, viscosity, stability and sterility.

In embodiments the formulation is provided or packaged in unit dosage form.

The invention encompasses all combinations of the particular embodiments recited herein, as if each combination had been laboriously recited.

### BRIEF DESCRIPTION OF THE DRAWINGS

FIGS. 1A-C. Serpina3n administration can treat glaucomatous neurodegeneration, as demonstrated by intravitreal injection of a recombinant serpina3n protein (R&D Systems, catalog #4709-PI-010) in a mouse model of glaucoma. (A) Summarized data showing serpina3a treatment reduced RGC death. RGCs were immunolabeled by Brn3a. * P<0.05. (B) Comparative 3D images of optic nerves by tissue clearing and light-sheet microscopy showing reduced axonal beading and fragmentation in the serpina3n treated mice than control. Green, Thy 1-YFP-labeled axons. (C) Corresponding boxed areas from (B) in higher magnification.

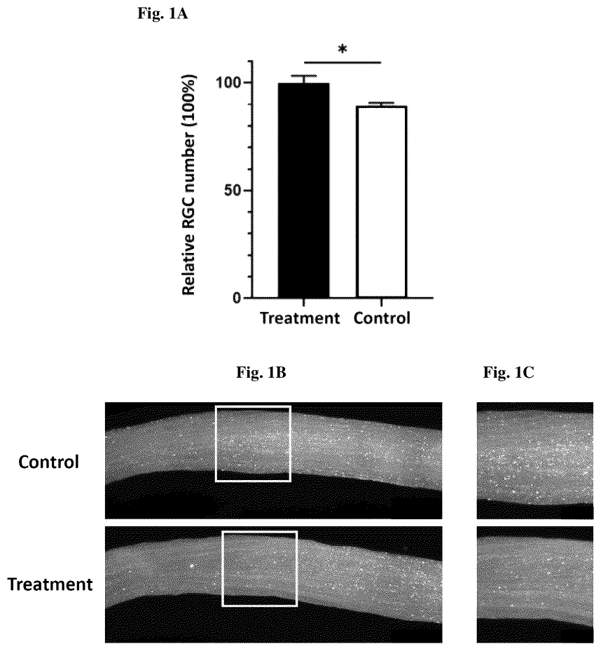

FIGS. 2A-B. Serpina3n can promote RGC survival and axonal growth, as demonstrated by in vitro RGC culture. RGCs isolated from mouse retinas were cultured in the presence of a recombinant serpina3n protein (R&D Systems, catalog #4709-PI-010) or control. Cells were immunolabeled for β III tubulin (green) and MAP-2 (red). A Immunostaining images showing serpina3n promoted RGC neurite outgrowth compared with the control condition. Blue: DAPI nuclear staining. (B) Summarized data from repetitive experiments. * P<0.05.

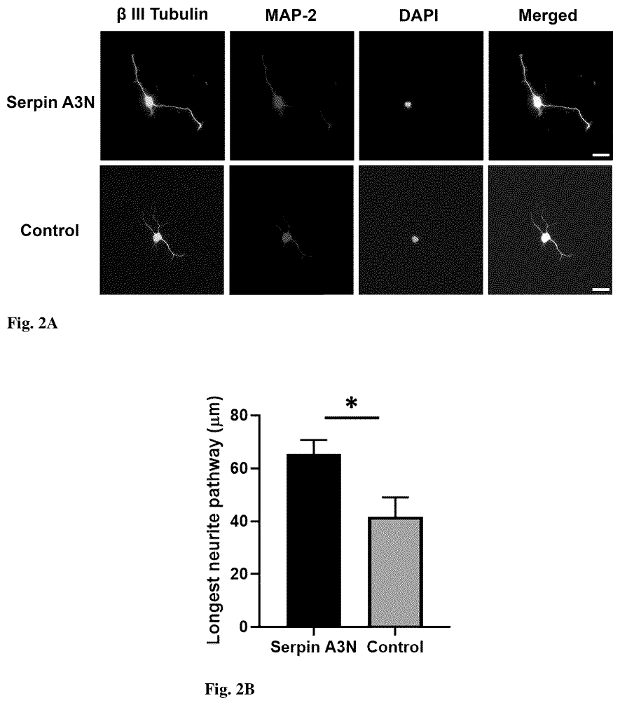

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

We demonstrate that the expression of serpinA3 increases in the optic nerve and mainly oligodendrocytes in glaucoma. Moreover, local or systemic (e.g. intravenous injection) administration of serpinA3 can treat glaucoma by reducing optic nerve damage and RGC death. These novel findings demonstrate serpinA3 modulation as a new therapeutic strategy to manage glaucoma by neuroprotection.

The disclosed methods and compositions can be used alone to prevent or treat glaucoma, or used in combination with other therapeutic approaches, such as eye drops, systemic medication, laser, and surgery to prevent or treat glaucoma.

### EXAMPLES

Increased expression of serpinA3 on the optic nerve in the glaucoma. In situ hybridization showed increased expression of serpina3n on the optic nerve in the glaucoma than normal condition, as demonstrated by a mouse model of glaucoma.

Differential expression of serpinA3 in oligodendrocytes of normal and glaucomatous conditions. Optic nerve single cell transcriptome high throughput RNA sequencing data showed differential expressional profiles of serpina3n in oligodendrocytes of normal and glaucomatous conditions, as demonstrated by a mouse model of glaucoma. Serpina3n expression level and the number of serpina3n positive cells were increased in the glaucomatous oligodendrocytes than normal control.

Treatment 1. SerpinA3 administration can treat glaucomatous neurodegeneration, as demonstrated by local intravitreal injection of a recombinant serpina3n (murine ortholog of human serpinA3) protein. Serpina3n treatment reduces RGC death. Comparative 3D images of optic nerves by tissue clearing and light-sheet microscopy show reduced axonal beading and fragmentation in the serpina3n treated mice than control.

These experiments were performed using the well-established mouse model of glaucoma (laser-induced occlusion of episcleral veins): Zhang L, et al. Establishment and Characterization of an Acute Model of Ocular Hypertension by Laser-Induced Occlusion of Episcleral Veins. Invest Ophthalmol Vis Sci. 2017 Aug. 1; 58(10):3879-3886). 0.2 ug recombinant mouse serpinA3N Protein (R&D Systems, Minneapolis, MN; 4709-PI-010) in 1 ul volume was administered by intravitreal injection on day 3 post the laser procedure to induce glaucoma.

Treatment 2. SerpinA3 administration can treat glaucomatous neurodegeneration, as demonstrated by local intravitreal injection of an adeno-associated viral (AAV)-serpinA3 vector. In this example, we demonstrate using intravitreal injection of 1 ul of the AAV-serpinA3 vector (5.0×10\hat{ }9 vg/uL), substantially as described in: Lee et al., IOVS 59(13)5398, November 2018; and Smith et al, Scientific Reports (2018) 8:1490; DOI:10.1038/s41598-018-19969-9. Administration of recombinant serpinA3N by AAV delivery also reduced the infarct size and improved motor function, associated with alleviated inflammation and oxidative stress in an ischemic stroke model; see, e.g. Zhang et al, CNS Neurosci Ther. 2022; 28:566-579

Treatment 3. SerpinA3 administration can treat glaucomatous neurodegeneration, as demonstrated by delivery of a recombinant serpina3n protein by systemic administration (intravenous injection) and by local administration (eyedrops). This treatment reduces RGC death. Comparative 3D images of optic nerves by tissue clearing and light-sheet microscopy show reduced axonal beading and fragmentation in the serpina3n treated mice than control. For systemic administration example, we selected for administration 50 ug recombinant mouse serpinA3N Protein (R&D Systems, 4709-PI-010) in 200 ul volume by tail vein on day 3 post laser to induce glaucoma. Our topical (eye drop) administration protocol was adapted from Hu et al., J Ocul Pharmacol Ther. 2016 May 1; 32(4): 203-210. Briefly, to formulate the serpinA3 eye drop solution, 5 mg of recombinant serpinA3 are reconstituted in 200 μL of sterile deionized water to create a final solution of 25 mg/mL. Each 10 μL eye drop consists of 9 μL, of the 25 mg/mL serpinA3 solution, and 1 μL of a 10% solution of the permeation enhancer saponin (Catalog #8047-15-2; Sigma) dissolved in sterile phosphate-buffered saline (PBS).

Treatment 4. SerpinA3 treatment can promote RGC survival and axonal growth. Using an in vitro cell culture we demonstrate that human serpinA3 or murine SerpinA3n proteins (R&D Systems) can promote retinal ganglion cell survival and axonal growth.

1. A method of treating glaucoma, comprising administering to an eye in need thereof a serpinA3 polypeptide or a serpinA3 polypeptide-encoding nucleic acid.

2. The method of claim 1, wherein the administering step comprises delivering the serpinA3 polypeptide.

3. The method of claim 1, wherein the administering step comprises delivering the serpinA3 polypeptide-encoding nucleic acid.

4. The method of claim 1, wherein the administering is systemically administering.

5. The method of claim 1, wherein the administering is locally administering.

6. The method of claim 1, wherein the administering step comprises delivery by topical, intracameral, subconjunctival, subretinal, intrascleral, intravitreal, or retrobulbar injection.

7. The method of claim 1, wherein the administration step comprises topical delivery in an ophthalmic gel, ointment, suspension or solution.

8. The method of claim 1, wherein the administration step comprises delivering recombinant serpinA3 by intravenous injection.

9. The method of claim 1, wherein the administration step comprises delivering recombinant serpinA3 administered by intravitreal injection.

10. The method of claim 1, wherein the administration step comprises delivering an adeno-associated viral (AAV)-serpinA3 vector by intravitreal injection.

11. The method of claim 1, wherein the administration step comprises delivering recombinant serpinA3 by eye drop.

12. The method of claim 1, further comprising treating the eye, or a person comprising the eye, a second, different glaucoma treatment, selected from eye drops, systemic medication, laser, and surgery to prevent or treat glaucoma.

13. The method of claim 1, further comprising detecting a resultant amelioration of the glaucoma.

14. The method of claim 1, further comprising detecting a resultant amelioration of the glaucoma by a decrease in retinal ganglion cell (RGC) death or axonal damage.

15. The method of claim 6, further comprising detecting a resultant amelioration of the glaucoma by a decrease in retinal ganglion cell (RGC) death or axonal damage.

16. The method of claim 7, further comprising detecting a resultant amelioration of the glaucoma by a decrease in retinal ganglion cell (RGC) death or axonal damage.

17. The method of claim 9, further comprising detecting a resultant amelioration of the glaucoma by a decrease in retinal ganglion cell (RGC) death or axonal damage.

18. The method of claim 10, further comprising detecting a resultant amelioration of the glaucoma by a decrease in retinal ganglion cell (RGC) death or axonal damage.

19. The method of claim 11, further comprising detecting a resultant amelioration of the glaucoma by a decrease in retinal ganglion cell (RGC) death or axonal damage.

20. An ophthalmic formulation of a serpinA3 polypeptide or a nucleic acid encoding the serpinA3 polypeptide, configured for treating glaucoma, wherein:
in the form of a topical ophthalmic gel, ointment, suspension or solution;
the dosage form is a serpinA3-loaded contact lens, eye drop, depot or bollus;
packaged in an eye drop dispenser;
loaded in a syringe configured for intracameral, subconjunctival, subretinal, intrascleral, intravitreal, or retrobulbar injection; and/or
further comprising excipients and features suitable for direct, topical delivery to the eye, selected from the group consisting of opthalmically suitable clarity, pH buffer, tonicity, viscosity, stability and sterility; and/or in unit dosage form.
